(19) United States
(12) Patent Application Publication
(10) Pub. No.: US 2025/0151875 A1
(43) Pub. Date: May 15, 2025

(54) Sunscreen Powder Kit

(71) Applicants: Kelly Roscamp, Lakeside, CA (US); Melissa Moore, Lakeside, CA (US)

(72) Inventors: Kelly Roscamp, Lakeside, CA (US); Melissa Moore, Lakeside, CA (US)

(21) Appl. No.: 18/388,241

(22) Filed: Nov. 9, 2023

(51) Int. Cl. A45D 33/00; A46B 5/02; A46B 9/02; B65D 43/02

(52) U.S. Cl. CPC A45D 33/003; A46B 5/021; A46B 9/028; B65D 43/0231; A46B 2200/1093; B65D 2543/00092; B65D 2543/00537

(57) ABSTRACT

A sunscreen powder kit includes a container which defines a containment space and a lid is removably attachable to the container for closing the containment space. A sunscreen powder is contained in the containment space in the container and the sunscreen powder comprises powdered zinc oxide to block ultraviolet light and powdered titanium dioxide to block ultraviolet light and powdered bamboo to stabilize the sunscreen powder during application. An application brush is provided which has a handle and a plurality of bristles each extending away from the handle. The handle can be gripped by a user thereby facilitating the user to place the plurality of bristles into the sunscreen powder for applying the sunscreen powder onto livestock to protect the livestock from sunburn.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to sunscreen devices and more particularly pertains to a new sunscreen device for protecting exposed skin of livestock from being sunburned. The device includes a container which contains a sunscreen powder which is comprised of powered zinc oxide and powdered titanium dioxide and powdered bamboo. The device includes an application brush for applying the sunscreen powder to exposed skin of livestock to protect the exposed skin from being sunburned.

### (2) Description of Related Art Including Information Disclosed Under 37 CFR 1.97 and 1.98

The prior art relates to sunscreen devices including a variety of topical sunscreens which each at least includes zinc oxide and titanium dioxide and a variety of dry sunscreens which each at least includes powdered zinc oxide and powdered titanium dioxide and in at least one instance includes siliceous compounds and diatomaceous algae. In no instance does the prior art disclose a sunscreen powder that is comprised of powdered zinc oxide and powdered titanium dioxide and powdered bamboo to stabilize the sunscreen powder.

### BRIEF SUMMARY OF THE INVENTION

An embodiment of the disclosure meets the needs presented above by generally comprising a container which defines a containment space and a lid is removably attachable to the container for closing the containment space. A sunscreen powder is contained in the containment space in the container and the sunscreen powder comprises powdered zinc oxide to block ultraviolet light and powdered titanium dioxide to block ultraviolet light and powdered bamboo to stabilize the sunscreen powder during application. An application brush is provided which has a handle and a plurality of bristles each extending away from the handle. The handle can be gripped by a user thereby facilitating the user to place the plurality of bristles into the sunscreen powder for applying the sunscreen powder onto livestock to protect the livestock from sunburn.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a sunscreen powder kit according to an embodiment of the disclosure.

FIG. 2 is a schematic view of an embodiment of the disclosure.

FIG. 3 is a perspective view of an embodiment of the disclosure showing an application brush being dipped into a sunscreen powder.

FIG. 4 is a perspective in-use view of an embodiment of the disclosure showing a sunscreen powder being applied to skin of livestock.

FIG. 5 is a perspective in-use view of an embodiment of the disclosure showing a measured amount of water being mixed with a measured amount of sunscreen powder to form a paste.

### DETAILED DESCRIPTION OF THE INVENTION

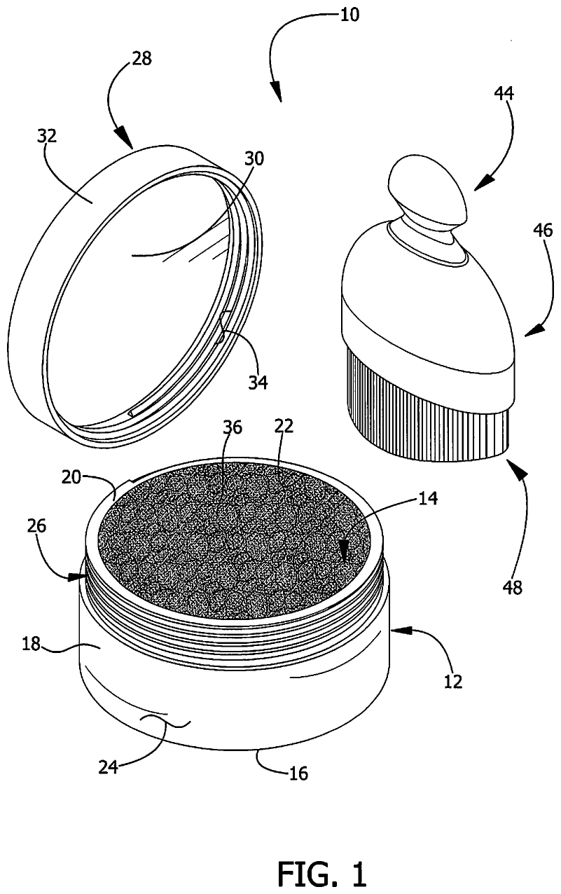

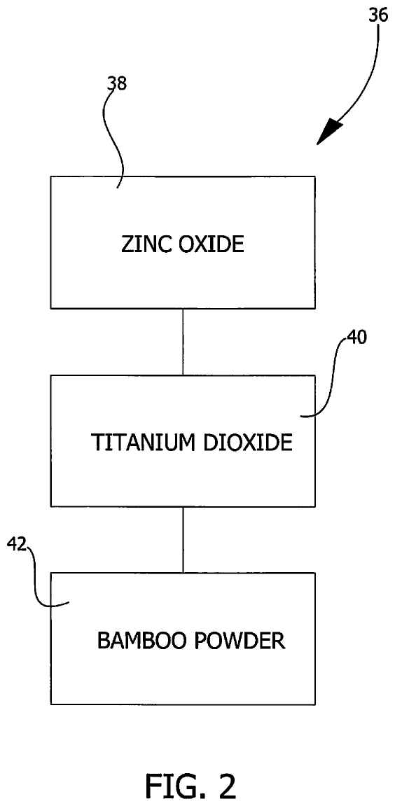

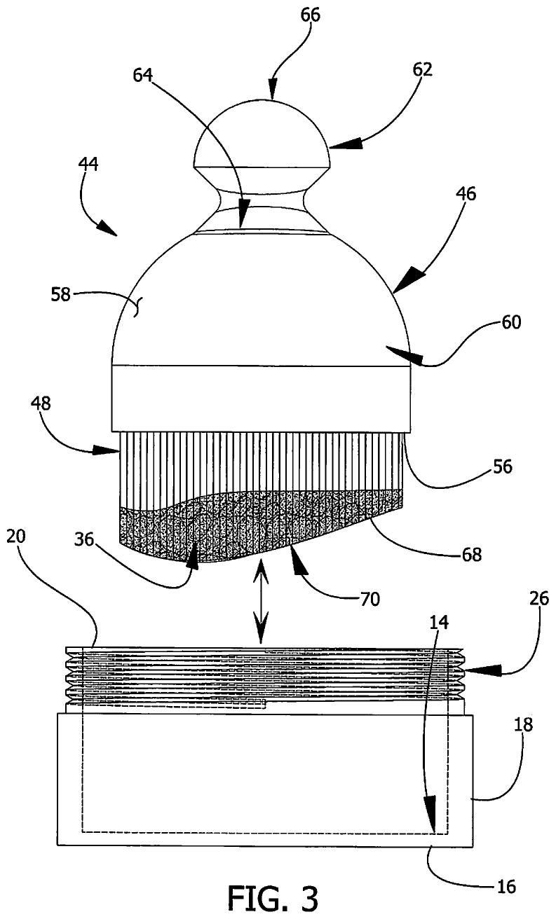

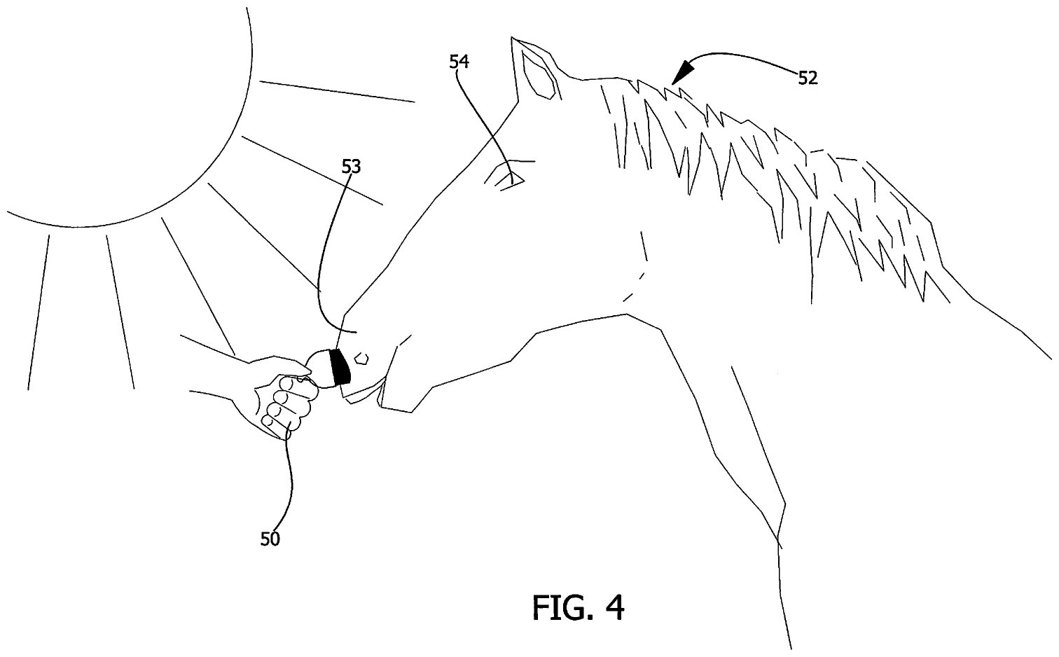

With reference now to the drawings, and in particular to FIGS. 1 through 5 thereof, a new sunscreen device embodying the principles and concepts of an embodiment of the disclosure and generally designated by the reference numeral 10 will be described.

As best illustrated in FIGS. 1 through 5, the sunscreen powder kit 10 generally comprises a container 12 which defines a containment space 14. The container 12 has a bottom wall 16 and a perimeter wall 18 extending upwardly from the bottom wall 16; the perimeter wall 18 is continuously arcuate about a center of the bottom wall 16 such that the container 12 has a cylindrical shape. The perimeter wall 18 has an upper edge 20 which defines an opening 22 into the containment space 14 defined by the container 12. The perimeter wall 18 has an outer surface 24; the outer surface 24 has a recessed portion 26 extending downwardly from the upper edge 20 toward the bottom wall 16 and the recessed portion 26 is threaded.

A lid 28 is provided and the lid 28 is removably attachable to the container 12 for closing the containment space 14. The lid 28 has an upper wall 30 and an outside wall 32 extending downwardly from the upper wall 30 and the outside wall 32 is continuously arcuate about a center of the upper wall 30 such that the lid 28 has a disk shape. The outside wall 32 has an inner surface 34 that is threaded and the inner surface 34 threadably engages the recessed portion 26 of the outer surface 24 of the perimeter wall 18 of the container 12 for securing the lid 28 to the container 12. The container 12 may have a fluid capacity ranging between approximately 80.0 ml and 180.0 ml.

A sunscreen powder 36 is contained in the containment space 14 in the container 12 and the sunscreen powder 36 comprises powdered zinc oxide 38 to block ultraviolet A and ultraviolet B radiation. The sunscreen powder 36 comprises a powdered titanium dioxide 40 to block ultraviolet A and ultraviolet B radiation. Furthermore, the sunscreen powder 36 comprises powdered bamboo 42 to stabilize the sunscreen powder 36 during application. The powdered bamboo 42 inhibits the sunscreen powder 36 from forming a dust thereby reducing the likelihood that the sunscreen powder 36 will become airborne and be subsequently inhaled by a user 50. The powdered zinc oxide 38 comprises 45.0 percent of the sunscreen powder 36 and the powdered titanium dioxide 40 comprises 45.0 percent of the sunscreen powder 36. Finally, the powdered bamboo 42 comprises 10.0 percent of the sunscreen powder 36.

An application brush 44 is provided which has a handle 46 and a plurality of bristles 48 each extending away from the handle 46. The handle 46 can be gripped by a user 50 thereby facilitating the user 50 to place the plurality of bristles 48 into the sunscreen powder 36 for applying the sunscreen powder 36 onto livestock 52 to protect the livestock 52 from sunburn. The livestock 52 may be horses, cattle, pigs or any other type of livestock that is commonly outdoors and which have patches of exposed skin 53 that is prone to being sunburned. Furthermore, the sunscreen powder 36 can be applied around eyes 54 of the livestock 52 without running into the livestock's eyes 54 and irritating the livestock's eyes 54 as can occur with a fluid sunscreen.

The handle 46 has a bottom surface 56 and an outside surface 58 extending away from the bottom surface 56 and the outside surface 58 has a lower portion 60 and an upper portion 62. The lower portion 60 curves upwardly from the bottom surface 56 toward an apex 64 of the lower portion 60 such that the lower portion 60 has a dome shape. The upper portion 62 curves outwardly from the apex 64 of the lower portion 60 and the upper portion 62 curves inwardly at an apex 66 of the upper portion 62 such that the upper portion 62 defines a rounded knob which can be gripped. Each of the plurality of bristles 48 is attached to and extends away from the bottom surface 56 of the handle 46. The plurality of bristles 48 has a decreasing length with respect to a lateral axis of the handle 46 such that a distal end 68 of each of the plurality of bristles 48 defines a sloped surface 70 with respect to the bottom surface 56 of the handle 46.

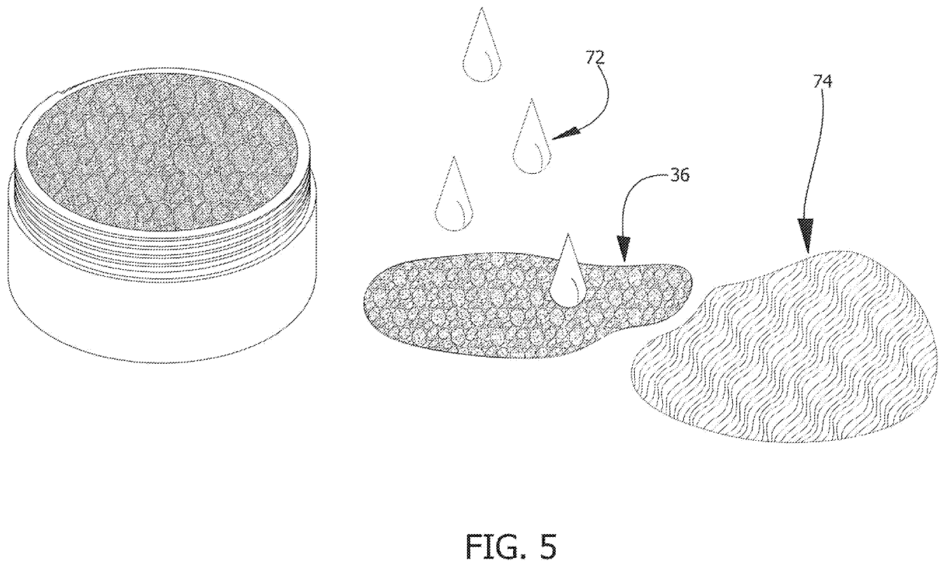

In use, the bristles 48 are inserted into the container 12 to coat the bristles 48 with the sunscreen powder 36 and the application brush 44 is manipulated to apply the sunscreen powder 36 to any areas of exposed skin 53 of the livestock 52. In this way the sunscreen powder 36 can protect the exposed skin 53 of the livestock 52 from being sunburned. Additionally, powdered bamboo 42 is known to have antibacterial properties as well as skin soothing properties thereby enhancing the health of the livestock's skin 53. The sunscreen powder 36 can be applied around the livestock's eyes 54 without the risk of the sunscreen powder 36 running into the livestock's eyes 54 as can occur with a liquid sunscreen. In this way the livestock 52 can remain comfortable while enjoying the benefits of sunscreen on their exposed skin. As is shown in FIG. 5, a measured amount of the sunscreen powder 36 can be removed from the container 12 and a measured amount of water 72 can be mixed with the measured amount of sunscreen powder 36 to form a paste 74. The paste 74 can be applied to the livestock's exposed skin 53 in lieu of the sunscreen powder 36 if so desired by the user 50.

With respect to the above description then, it is to be realized that the optimum dimensional relationships for the parts of an embodiment enabled by the disclosure, to include variations in size, materials, shape, form, function and manner of operation, kit and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A sunscreen powder kit for applying to livestock in order to protect the livestock from sunburn, said kit comprising:
a container defining a containment space;
a lid being removably attachable to said container for closing said containment space;
a sunscreen powder being contained in said containment space in said container, said sunscreen powder comprising powdered zinc oxide wherein said powdered zinc oxide is configured to block ultraviolet A and ultraviolet B radiation, said sunscreen powder comprising a powdered titanium dioxide wherein said powdered titanium dioxide is configured to block ultraviolet A and ultraviolet B radiation, said sunscreen powder comprising powdered bamboo to stabilize said sunscreen powder during application; and
an application brush having a handle and a plurality of bristles each extending away from said handle wherein said handle is configured to be gripped by a user thereby facilitating the user to place said plurality of bristles into said sunscreen powder for applying the sunscreen powder onto livestock to protect the livestock from sunburn.

2. The kit according to claim 1, wherein:
said container has a bottom wall and a perimeter wall extending upwardly from said bottom wall, said perimeter wall being continuously arcuate about a center of said bottom wall such that said container has a cylindrical shape, said perimeter wall having an upper edge defining an opening into said containment spaced defined by said container, said perimeter wall having an outer surface, said outer surface having a recessed portion extending downwardly from said upper edge toward said bottom wall, said recessed portion being threaded; and
said lid has an upper wall and an outside wall extending downwardly from said upper wall, said outside wall being continuously arcuate about a center of said upper wall such that said lid has a disk shape, said outside wall having an inner surface being threaded, said inner surface threadably engaging said recessed portion of said outer surface of said perimeter wall of said container for securing said lid to said container.

3. The kit according to claim 1, wherein said powdered zinc oxide comprises 45.0 percent of said sunscreen powder.

4. The kit according to claim 1, wherein said powdered titanium dioxide comprises 45.0 percent of said sunscreen powder.

5. The kit according to claim 1, wherein said powdered bamboo comprises 10.0 percent of said sunscreen powder.

6. The kit according to claim 1, wherein:
said handle has a bottom surface and an outside surface extending away from said bottom surface;
said outside surface has a lower portion and an upper portion;
said lower portion curves upwardly from said bottom surface toward an apex of said lower portion such that said lower portion has a dome shape; and
said upper portion curves outwardly from said apex of said lower portion and said upper portion curves inwardly at an apex of said upper portion such that said upper portion defines a rounded knob wherein said rounded knob is configured to be gripped.

7. The kit according to claim 6, wherein:
each of said plurality of bristles is attached to and extends away from said bottom surface of said handle; and
said plurality of bristles has a decreasing length with respect to a lateral axis of said handle such that a distal end of each of said plurality of bristles defines a sloped surface with respect to said bottom surface of said handle.

8. A sunscreen powder kit for applying to livestock in order to protect the livestock from sunburn, said kit comprising:
a container defining a containment space, said container having a bottom wall and a perimeter wall extending upwardly from said bottom wall, said perimeter wall being continuously arcuate about a center of said bottom wall such that said container has a cylindrical shape, said perimeter wall having an upper edge defining an opening into said containment spaced defined by said container, said perimeter wall having an outer surface, said outer surface having a recessed portion extending downwardly from said upper edge toward said bottom wall, said recessed portion being threaded;
a lid being removably attachable to said container for closing said containment space, said lid having an upper wall and an outside wall extending downwardly from said upper wall, said outside wall being continuously arcuate about a center of said upper wall such that said lid has a disk shape, said outside wall having an inner surface being threaded, said inner surface threadably engaging said recessed portion of said outer surface of said perimeter wall of said container for securing said lid to said container;
a sunscreen powder being contained in said containment space in said container, said sunscreen powder comprising powdered zinc oxide wherein said powdered zinc oxide is configured to block ultraviolet A and ultraviolet B radiation, said sunscreen powder comprising a powdered titanium dioxide wherein said powdered titanium dioxide is configured to block ultraviolet A and ultraviolet B radiation, said sunscreen powder comprising powdered bamboo to stabilize said sunscreen powder during application, said powdered zinc oxide comprising 45.0 percent of said sunscreen powder, said powdered titanium dioxide comprising 45.0 percent of said sunscreen powder, said powdered bamboo comprising 10.0 percent of said sunscreen powder; and
an application brush having a handle and a plurality of bristles each extending away from said handle wherein said handle is configured to be gripped by a user thereby facilitating the user to place said plurality of bristles into said sunscreen powder for applying the sunscreen powder onto livestock to protect the livestock from sunburn, said handle having a bottom surface and an outside surface extending away from said bottom surface, said outside surface having a lower portion and an upper portion, said lower portion curving upwardly from said bottom surface toward an apex of said lower portion such that said lower portion has a dome shape, said upper portion curving outwardly from said apex of said lower portion and said upper portion curving inwardly at an apex of said upper portion such that said upper portion defines a rounded knob wherein said rounded knob is configured to be gripped, each of said plurality of bristles being attached to and extending away from said bottom surface of said handle, said plurality of bristles having a decreasing length with respect to a lateral axis of said handle such that a distal end of each of said plurality of bristles defines a sloped surface with respect to said bottom surface of said handle.

9. A method of applying sunscreen to livestock in a manner that inhibits sunscreen from running on the livestock's skin, the steps of the method comprising:
exposing a sunscreen which is contained in a container, said sunscreen comprising a mixture of powdered zinc oxide and powdered titanium dioxide and powdered bamboo;
inserting bristles of an application brush into said container thereby facilitating said bristles to be coated with said sunscreen; and
applying said sunscreen to skin of livestock with said application brush wherein said sunscreen is configured to protect the skin of the livestock from being sunburned.

10. The method according to claim 9, wherein said sunscreen is a powder.

11. The method according to claim 10, wherein said powder is applied directly to the skin of the livestock.

12. The method according to claim 10, wherein the steps of the method further comprise:
removing a measured amount of said powder from said container;
adding water to said measured amount of said powder;
mixing said water and said measured amount of powder to create a paste; and
applying said paste to skin of the livestock wherein said paste is configured to protect the skin of the livestock from being sunburned.
